(19) United States
(12) Patent Application Publication
(10) Pub. No.: US 2002/0087174 A1
(43) Pub. Date: Jul. 4, 2002

(54) Mesh for surgical use

(75) Inventor: Giovanni Capello, Torino (IT)

Correspondence Address: SUGHRUE, MION, ZINN,, MACPEAK & SEAS, PLLC, 2100 Pennsylvania Avenue, N.W., Washington, DC, 20037-3213, US

(73) Assignee: SOVETA S.R.L.

(21) Appl. No.: 10/026,552

(22) Filed: Dec. 27, 2001

(30) Foreign Application Priority Data
Dec. 28, 2000 (IT) T02000U000230

(51) Int. Cl. A61B 17/08

(52) U.S. Cl. 606/151

(57) ABSTRACT

A radio-opaque surgical mesh comprising polyfilaments containing 60% of barium sulphate is described.

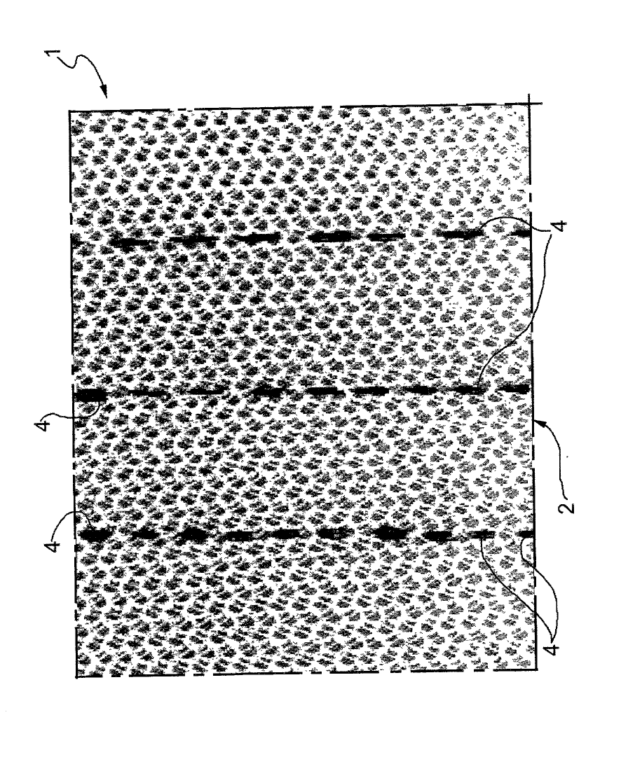

### DESCRIPTION

The present invention relates to a mesh for surgical use.

A great variety of surgical meshes, such as, for example, those used for hernia surgery or for containment surgery in general, are used in surgery.

These meshes are well known in the medical field and have the disadvantage that, if they move from the optimal intended position, the surgeon has no option other than to operate on the patient again in order to see whether, at what point, and how the meshes have moved.

The object of the present invention is to provide a mesh for surgical use which can be located if necessary, without the need to operate on the patient again, simply with the use of radiological techniques.

The present invention achieves the objects specified by means of a mesh for surgical use having the specific characteristics recited in the appended claims.

Further characteristics and advantages of the present invention will become clear in the course of the following detailed description which is given purely by way of nonlimiting example with reference to the appended drawing.

In the drawing, a mesh for surgical use according to the present invention is indicated 1 and comprises a mesh element 2 of known type which comprises radio-opaque elements 4, as will be explained further below.

The surgical mesh 2 is normally made of synthetic polypropylene monofilaments, whereas the radio-opaque element is formed by a filament or a polyfilament containing 60% of barium sulphate, woven into or otherwise connected to the mesh 2.

The mesh of the present invention can be rendered fully or partially radio-opaque, in dependence on the number of elements 4 present.

Barium sulphate is a product which is used surgically, can be implanted in the organism in stable manner, and can be moulded to various shapes and extents by means of an electro-coagulator; up to now—with the use of conventional surgical meshes—in the event of migration or incorrect positioning of the mesh prosthesis, the surgeon had no option other than to operate on the patient again to check the state and the position of the prosthesis.

By virtue of the radio-opaque surgical mesh according to the invention, the surgeon can follow the prosthesis radiographically during the postoperative period and can determine its precise anatomical position and its correct arrangement, that is to say, perfect stretching-out of the anatomical surfaces without pleating, with the use of the radiological technique, since barium sulphate—as already stated—is opaque to X-rays.

The advantages of a surgical mesh according to the invention are clear from the foregoing description, since it drastically reduces the number of repeat operations, with clear advantages to the patient and, not least, with a considerable reduction in hospitalization costs.

The mesh of the present invention can also easily be pre-shaped according to the uses for which it is intended.

What is claimed is:

1. Mesh for surgical use, characterized in that it is at least partially radio-opaque.

2. Mesh for surgical use according to claim 1, characterized in that it comprises barium sulphate filaments or polyfilaments.

3. Mesh for surgical use according to claim 2, characterized in that the barium sulphate is at a concentration of about 60%.

4. Mesh for surgical use according to claim 1, characterized that it comprises synthetic polypropylene monofilaments.
